(19) United States
(12) Patent Application Publication
(10) Pub. No.: US 2008/0161920 A1
(43) Pub. Date: Jul. 3, 2008

(54) Dynamizing Interbody Implant and Methods for Stabilizing Vertebral Members

(75) Inventor: Anthony J. Melkent, Memphis, TN (US)

Correspondence Address: COATS & BENNETT, PLLC, 1400 Crescent Green, Suite 300, Cary, NC, 27518, US

(73) Assignee: WARSAW ORTHOPEDIC, INC., Warsaw, IN (US)

(21) Appl. No.: 11/538,190

(22) Filed: Oct. 3, 2006

(51) Int. Cl. A61F 2/44

(52) U.S. Cl. 623/17.11

(57) ABSTRACT

The application is directed to implants and methods of spacing vertebral members. The implants include a body sized to fit within the desired space. One or more relief cuts are formed within the body and may include a variety of sizes, and shapes. Resorbable material is positioned within the relief cuts. The resorbable material includes an initial stiffness to give the implant a first rigidity when initially inserted into the patient. Over time, the resorbable material is absorbed causing the stiffness to decrease thereby lessening the rigidity of the implant. In general, the implant includes a maximum rigidity when the implant is initially inserted within the patient, and a minimum rigidity after the resorbable material is absorbed within the patient.

### BACKGROUND

The present application is directed to implants and methods for dynamic stabilization and/or fusion of vertebral members and, more specifically, to implants with resorbable material that is absorbed by the patient and changes the stiffness of the implant.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Various conditions may lead to damage of the intervertebral discs and/or the vertebral members. The damage may result from a variety of causes including a specific event such as trauma, a degenerative condition, a tumor, or infection. Damage to the intervertebral discs and vertebral members can lead to pain, neurological deficit, and/or loss of motion.

Implants may be positioned between the vertebral members to stabilize the spine. The implants may also replace an entirety or a section of a vertebral member, the entirety or a section of an intervertebral disc, or both. Implants may also provide support and stabilization without removing the damaged vertebral members or discs. The implants should reduce or eliminate the pain and neurological deficit.

### SUMMARY

The present application is directed to implants and methods for dynamic stabilization and/or fusion of vertebral members. The implants may include a body with one or more relief cuts that contain a resorbable material. During an initial period, the resorbable material affects the stiffness of the implant. As the resorbable material is absorbed, the stiffness of the implant decreases as the resorbable material has less effect. In one embodiment, the resorbable material is completely absorbed and the stiffness of the implant is based solely on the body.

### BRIEF DESCRIPTION OF THE DRAWINGS

FIG. 1 is a side view of an implant between vertebral members according to one embodiment.

FIG. 2 is a side view of an implant between vertebral members according to one embodiment.

FIG. 3 is a perspective view of an implant according to one embodiment.

FIG. 4 is a perspective view of an implant according to one embodiment.

FIG. 5 is a perspective view of an implant according to one embodiment.

FIG. 6 is a perspective view of an implant according to one embodiment.

FIG. 7 is side view of an implant according to one embodiment.

FIG. 8 is side view of an implant according to one embodiment.

FIG. 9 is side view of an implant according to one embodiment.

FIG. 10 is a perspective view of an implant according to one embodiment.

FIGS. 11A, 11B, and 11C are side views of an implant with a resorbable material that is absorbed according to one embodiment.

FIGS. 12A and 12B are sectional views of an implant with a resorbable material that is absorbed according to one embodiment.

### DETAILED DESCRIPTION

The present application is directed to implants and methods for dynamic stabilization and/or fusion of vertebral members. The implants include a body with relief cuts that extend from an exterior surface of the body inward to an interior region of the body. A resorbable material is positioned within the cuts. The resorbable material initially affects the stiffness of the implant. Over time, the resorbable material is absorbed that causes the stiffness of the implant to lessen. In one embodiment, resorbable material is completely absorbed and the stiffness of the implant becomes that of the body.

By designing the cuts a certain way, there could be intermediate stiffnesses that occur between the initial implantation time (maximum stiffness) and the final minimum stiffness timepoint. This can be accomplished by having cuts that contribute different stiffnesses to the implant (i.e. different spring rates) and using resorbable material with different absorbtion rates in the different cuts, or by using smaller or larger cuts that have different amounts of resorbable material thus affecting the absorption rate. It could also be accomplished by using the same material in these different cuts, but having more or less surface area of the resorbable material exposed, thus affecting the absorption rate. This can be done by creating channels, holes, or perforations in the body to allow more of the resorbable material of a certain cut to be exposed. Intermediate stiffnesses may also be accomplished by positioned two or more different types of materials within the cuts.

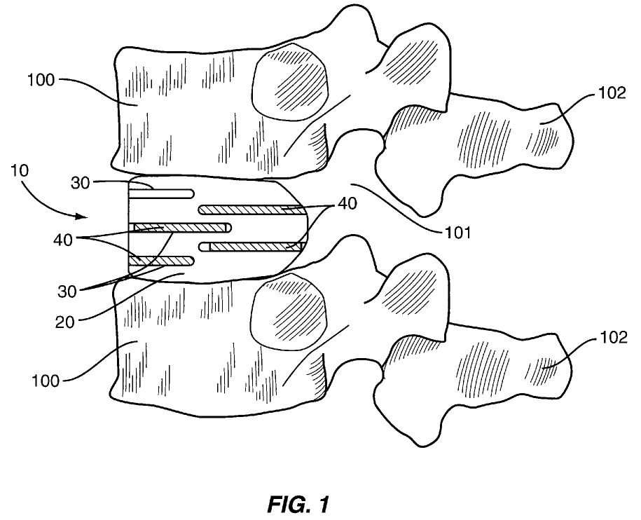

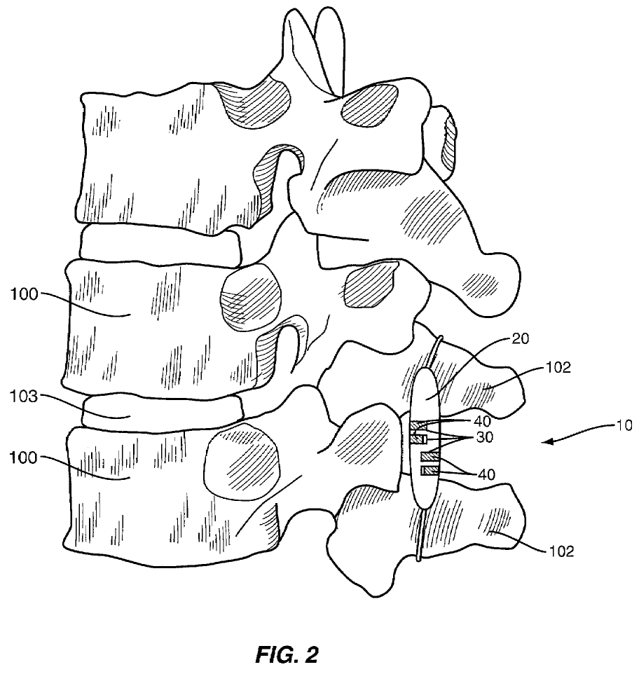

FIGS. 1 and 2 illustrate embodiments of implants 10. FIG. 1 includes the implant 10 positioned within the intervertebral space 101 formed between vertebral members 100. FIG. 2 includes an interspinous implant 10 positioned between spinous processes 102. The implants 10 include a body 20 sized to fit within the desired space. Relief cuts 30 are formed within the body 20. The size, shape, and number of relief cuts 30 may vary depending upon the context of use. Resorbable material 40 is positioned within the relief cuts 30. The resorbable material 40 includes an initial stiffness to give the implant 10 a first stiffness when initially inserted into the patient. Over time, the resorbable material 40 is absorbed within the patient causing a decrease in the stiffness of the implant 10. In general, the implant 10 includes a maximum stiffness when initially inserted within the patient, and a minimum stiffness after complete absorption of the material 40.

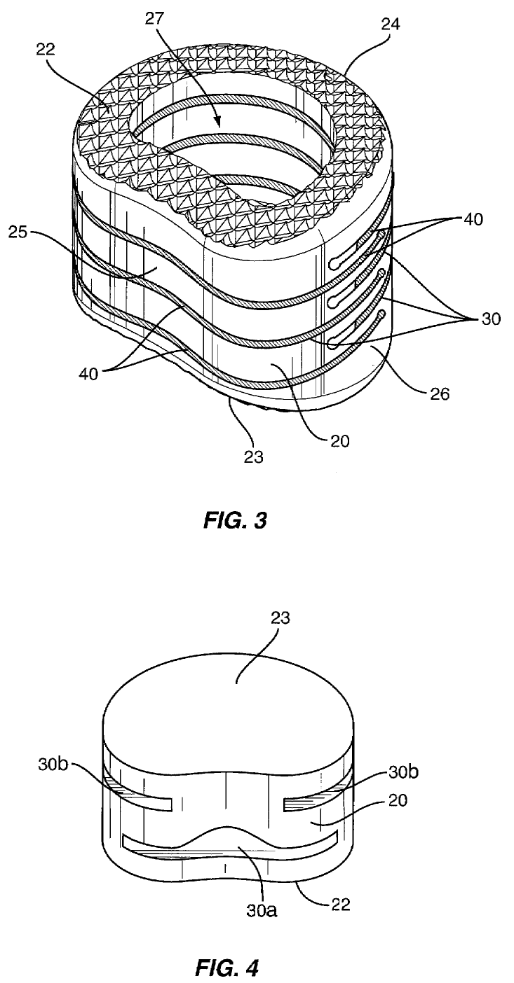

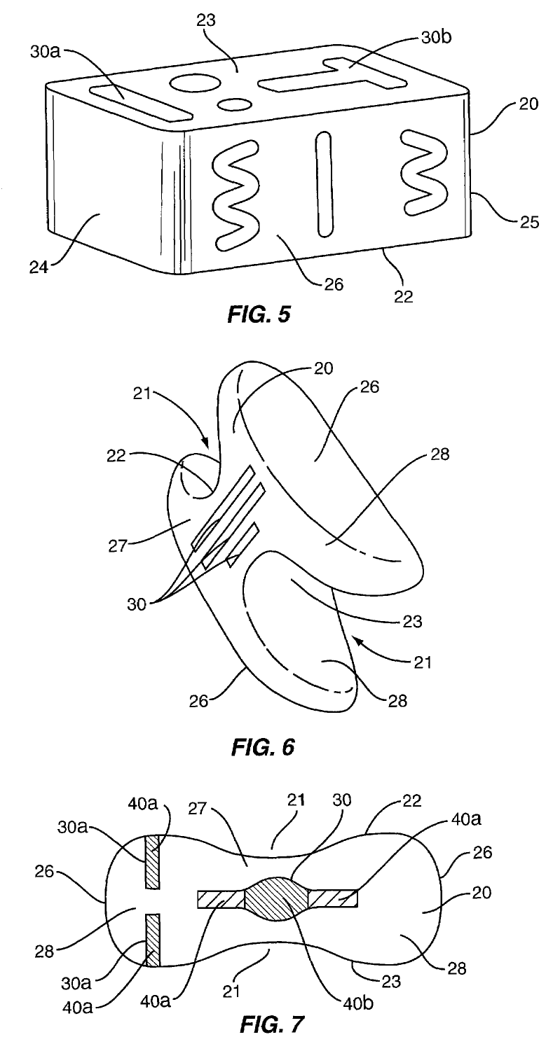

FIG. 3 illustrates an implant 10 that comprises a body 20 with relief cuts 30 that contain resorbable material 40. The body 20 is sized for positioning within the intervertebral space 101. Body 20 includes superior and inferior sides 22, 23 that each contact one of the vertebral members 100. The sides 22, 23 may include teeth for engaging the vertebral members 100. Body 20 further includes an anterior side 24, posterior side 25, and lateral sides 26. Other embodiments of the body 20 may include a variety of shapes and sizes. In one embodiment, the body 20 is substantially cylindrical with curved sides. FIG. 5 includes another embodiment with a substantially rectangular body 20. Further, the body 20 may be solid, or may include a hollow interior. Body 20 of FIG. 3 includes an aperture 27 extending through a central section to allow bone growth between the vertebral members 100. FIG. 4 illustrates an embodiment with a body 20 that does not include an aperture 27.

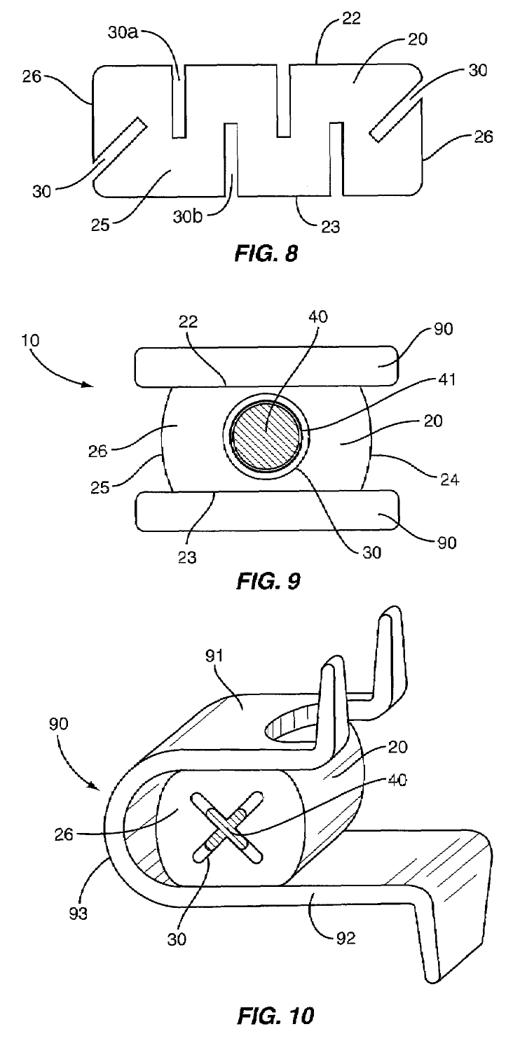

FIG. 6 illustrates a body 20 for spacing apart the spinous processes 102. This body 20 includes a core 27 with a pair of lateral wings 28 that together form channels 21 for receiving the spinous processes 102. FIG. 7 illustrates another embodiment of an interspinous body with less pronounced channels 21 formed by the core 27 and wings 28. FIG. 8 includes a body 20 for insertion in either the interspinous or intervertebral spaces.

Body 20 may further be positioned between one or more mounts 90. FIG. 9 includes an intervertebral implant 10 with a body 20 positioned between opposing mounts 90. Mounts 90 are sized and shaped to contact the vertebral members 100 and position the body 20 within the intervertebral space 101. Body 20 may include a variety of shapes and sizes and includes superior and inferior sides that contact the mounts 90. The embodiment illustrated in FIG. 9 is for use within the intervertebral space 101.

FIG. 10 illustrates another embodiment with a single mount 90 sized to receive the body 20. Mount 90 includes superior and inferior sections 91, 92 that contact the vertebral members 100. An intermediate section 93 extends between the sections 91, 92. Body 20 is sized to fit within the space formed by the sections 91, 92, 93. Body 20 may be connected to one or more of the sections 91, 92, 93 to maintain the position within the mount 90. This embodiment is constructed to fit within the interspinous space between the spinous processes 102.

Body 20 may be formed of a variety of materials. Embodiments feature materials such as metals suitable for surgical implants such as stainless steel, titanium, nickel titanium, and cobalt chromium. Body 20 may also be formed of bone. Polymer materials may also be used, including members of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE.

Body 20 may also be constructed of a substantially elastic material such as elastomeric materials, hydrogels or other hydrophilic-polymers, or composites thereof. Suitable elastomers include silicone, polyurethane, copolymers of silicone and polyurethane, polyolefins, such as polyisobutylene and polyisoprene, neoprene, nitrile, vulcanized rubber and combinations thereof. Suitable hydrogels include natural hydrogels, and those formed from polyvinyl alcohol, acrylamides such as polyacrylic acid and poly(acrylonitrile-acrylic acid), polyurethanes, polyethylene glycol, poly(N-vinyl-2-pyrrolidone), acrylates such as poly(2-hydroxy ethyl methacrylate) and copolymers of acrylates with N-vinyl pyrrolidone, N-vinyl lactams, acrylamide, polyurethanes and polyacrylonitrile, or may be other similar materials that form a hydrogel. The hydrogel materials may further be cross-linked to provide further strength to the implant. Examples of polyurethanes include thermoplastic polyurethanes, aliphatic polyurethanes, segmented polyurethanes, hydrophilic polyurethanes, polyether-urethane, polycarbonate-urethane and silicone polyether-urethane. Other suitable hydrophilic polymers include naturally-occurring materials such as glucomannan gel, hyaluronic acid, polysaccharides, such as cross-linked carboxyl-containing polysaccharides, and combinations thereof.

The body 20 may be constructed of a single material, or two or more combinations of materials. Further, the body 20 may include a substantially solid, uniform construction, or may include internal chambers or pores for receiving bone growth promoting material.

One or more relief cuts 30 are positioned within the body 20. The term “relief cuts” is used in a general sense to refer to spaces that extend from an exterior surface inward into an interior region of the body 20. Cuts 30 function to contain the resorbable material 40. Cuts 30 may also decrease the stiffness of the body 20. The number, size, and shape of the cuts 30 are each factors that affect the overall stiffness of the body 20, and the amount of possible deflection. By way of example, cuts 30 with greater heights may provide for greater amounts of deflection than smaller, narrower cuts 30. One embodiment of a body with relief cuts is disclosed in co-pending U.S. patent application Ser. No. 11/538,180 filed on the same day as the present application and entitled “Dynamic Devices and Methods for Stabilizing Vertebral Members”, herein incorporated by reference.

The design of the cuts 30 may affect the stiffness of the implant 10. The spring rate of the body 20 may be adjusted depending upon the size, shape, position, and number of cuts 30. Further, the size of the cut 30 may affect the amount of resorbable material 40 that may be placed within the cut 30. Further, the cuts 30 may be designed to have more or less surface area of the resorbable material 40 exposed, thus affecting the absorbtion rate.

The cuts 30 may include a depth that stops within an interior of the body 20, or may extend through the body 20. The shape and size of the cuts 30 may vary depending upon the context. Cuts 30 may also be substantially planar as illustrated in FIG. 3, or non-planar as illustrated in FIG. 9. Cuts 30 may include a height formed between opposing sidewalls. The heights may vary along the length (e.g., cut 30a of FIG. 4), or may be substantially constant (e.g. cuts 30 of FIG. 3).

In the various embodiments, the cuts 30 may overlap. FIG. 3 illustrates an embodiment with overlap between the superior and inferior sides 22, 23. FIG. 8 illustrates overlap between the lateral sides 26. The overlapped cuts 30 from different directions may be alternating as illustrated in FIGS. 1 and 3, or the cuts 30 may be arranged with two or more cuts from one direction grouped together as illustrated in FIG. 2.

Resorbable material 40 is positioned within the cuts 30 to affect the stiffness of the implant 10. Over time, the material 40 is gradually absorbed causing a decrease in the stiffness of the implant 10. This may further cause more load to be transferred to the developing fusion mass instead of being carried by the implant 10.

In one embodiment, material 40 works in combination with the body 20 to support the vertebral members 100 when the implant 10 is initially inserted within the patient. The body 20 with resorbable material 40 initially causes the implant 10 to have a first stiffness. As the material 40 is absorbed over time, the stiffness of the implant 10 decreases. This decrease loads the surrounding vertebral members 100 and stimulates more substantially bone mass. In general, the implant 10 has the greatest stiffness when initially inserted within the patient, and the least stiffness after absorption of the material 40. Intermediate levels of stiffness may occur during the time that the material 40 is being absorbed.

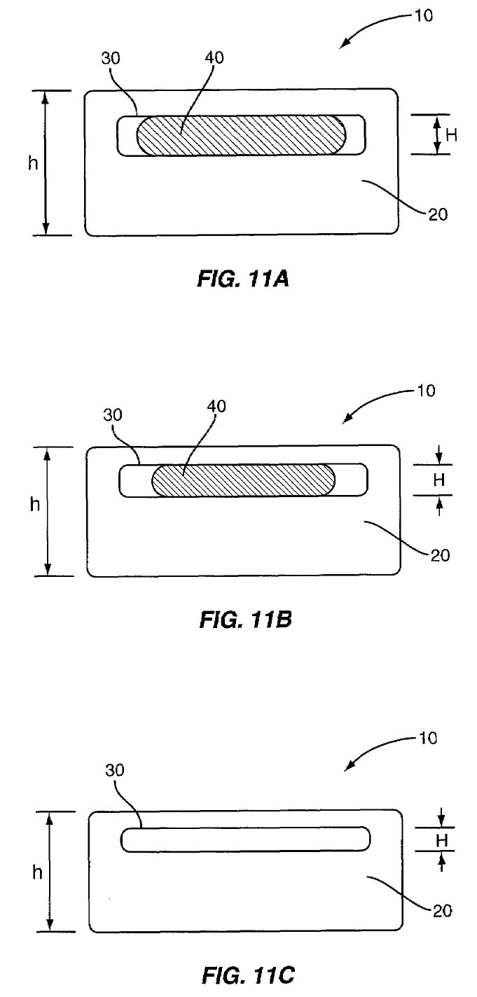

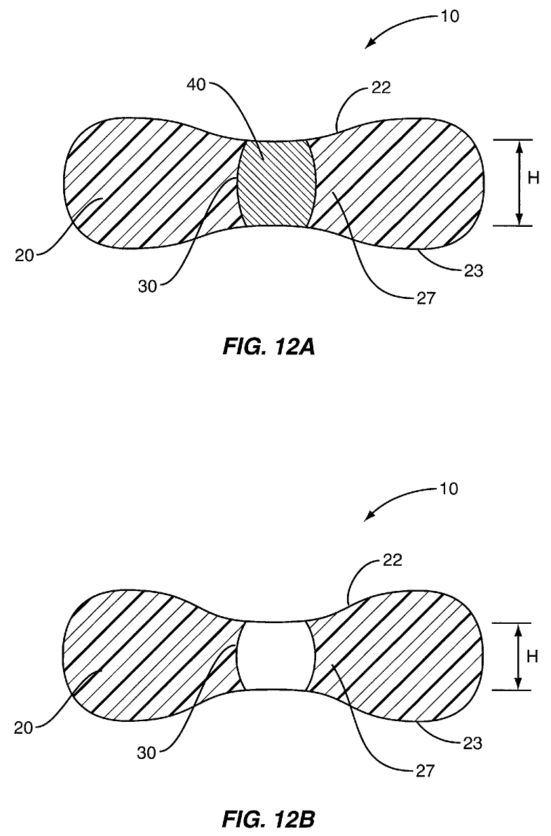

FIGS. 11A-11C illustrate one embodiment of the implant 10. As illustrated in FIG. 11A, the implant 10 includes a cut 30 that contains material 40. Upon being initially inserted into the patient, the cut 30 includes a height H. The rigidity of the implant 10 is caused by a combination of the body 20 and material 40. FIG. 11B illustrates the implant 10 after a time when the material 40 is partially absorbed. The rigidity of the implant 10 is still a combination of the body 20 and material 40. As the material 40 is absorbed, the body 20 accounts for a greater amount of the rigidity. The height H of the cut 30, and the height h of the implant 10 may be substantially the same as before, or may be less. FIG. 11C illustrates the implant 10 after the material 40 is completely absorbed and the rigidity of the implant 10 is based solely on the body 20. Both the height H of the cut 30 and height H of the body 20 are smaller than when the implant 10 was initially placed within the patient. In this embodiment, the material 40 is completely absorbed. In other embodiments, the material 40 may not become completely absorbed, with a residual amount remaining within the body 20.

When the material 40 absorbs as illustrated in FIGS. 11B and 11C, the load resistance of the implant 10 is decreased. This change in resistance may result in the compressive forces applied by the vertebral members 100 causing a decrease in the cut height H and body height h. This may further cause more load to be transferred to the vertebral members 100 instead of being carried by the implant 10.

The material 40 may also work alone to affect the initial stiffness of the implant 10. In one embodiment as illustrated in FIG. 12A, the cut 30 extends between the superior and inferior sides 22, 23. The material 40 within the cut 30 directly contacts the vertebral members 100. During an initial period, the rigidity of the implant 10 to support the vertebral members 100 is based solely on the material 40. As the material 40 is absorbed, the rigidity of the material 40 lessens causing the overall rigidity of the implant 10 to be a combination of the material 40 and body 20. When the material 40 is completely absorbed as illustrated in FIG. 12B, the rigidity of the implant 10 is based solely on the body 20.

The material 40 may completely or partially fill the cuts 30. FIG. 3 illustrates an embodiment with the cuts 30 being completely filled by the material 40. In another embodiment as illustrated in FIGS. 9 and 10, material 40 does not completely fill the cuts 30. In some embodiments with multiple cuts 30, material 40 is only positioned within some of the cuts 30 with the remainder being empty.

In one embodiment, the material 40 is inserted into the cuts 30 prior to the body 20 being inserted into the patient. In other embodiments, the material 40 is inserted into one or more of the cuts 30 after the body 20 is inserted into the patient.

Each of the cuts 30 may be filled by one type of material 40, or two or more of the cuts 30 may be filled with different materials 40. With multiple materials 40, the materials 40 may include different stiffnesses and/or a different absorption rate. In one multiple material embodiment as illustrated in FIG. 7, a first portion of the cut 30 is occupied by the hard, resorbable material 40a, and a second portion is occupied by a second elastic material 40b. As the resorbable material 40a is absorbed, the load is transferred through the elastic material 40b, providing an intermediate stiffness. As the resorbable material 40a is absorbed in the other cuts 30a that do not have the elastic material 40b, the stiffness shifts from that intermediate stiffness to another intermediate stiffness or the final stiffness, depending on the design. Another embodiment includes two or more of the cuts 30 with both materials 40a, 40b. As the resorbable material 40a is absorbed, the implant 10 transfers to a final stiffness that includes the stiffness of the combined body 20 and elastic material 40b.

Resorbable material may be formed from a wide variety of natural or synthetic materials. The material may be elastic or elastomeric, deformable, or non-compliant. Suitable resorbable materials include fibrin, albumin, collagen, elastin, silk and other proteins, polyethylene oxide, cyanoacrylate, polylactic acid, polyester, polyglycolic acid, polypropylene fumarate, tyrosine-based polycarbonate and combinations thereof. Other suitable materials include demineralized bone matrix. In one embodiment, resorbable material may be a woven fabric. One embodiment of a spacer with a resorbable material is disclosed in U.S. patent application Ser. No. 11/341,233 filed on Jan. 27, 2006, and entitled “Interspinous Devices and Methods of Use” which is hereby incorporated by reference.

The amount of time for the material to be absorbed into the patient may vary. In some embodiments, the absorption begins immediately upon insertion into the patient. In other embodiments, absorptions does not begin until a period of time after insertion. In one embodiment as illustrated in FIG. 9, a coating 41 is placed around the material 40 to delay the onset of absorption.

In one embodiment, the material 40 is loosely positioned within the cuts 30. In another embodiment, the material 40 is attached to the surface of the cuts 30. Cuts 30 may include surface features including chemical modifications and surface configurations that improve the bonding with the material 40. In one embodiment, the outer surface is chemically modified, such as by surface grafting, and pre-coating with a primer such as a layer of adhesive, sealant, or other like materials.

The implant 10 may be used as a motion-preserving device that maintains motion of the vertebral members 100. In this context, the implant 10 dynamically stabilizes the vertebral members 100 and allows for continued vertebral movement. The implant 10 may also be used as a fusion device that fuses together the vertebral members 100. In some embodiments, the implant 10 functions as both a dynamic motion-preserving device and a fusion device.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. An implant for spacing apart vertebral members of a patient, the implant comprising:
a body including an exterior surface and an interior region;
a relief cut contained within the body and including inferior and superior sides that extend from the exterior surface inward into the interior region; and
a resorbable material positioned within the relief cut, the resorbable material being absorbed within a predetermined amount of time;
a stiffness of the implant decreasing from a first level when initially inserted between the vertebral members when the resorbable material is within the relief cut, and a second lesser level after the predetermined period of time when the resorbable material has been absorbed within the patient.

2. The implant of claim 1, wherein the body is sized to fit within an intervertebral space and the exterior surface is shaped to contact the vertebral members.

3. The implant of claim 1, wherein the body is shaped to fit within an interspinous space and the exterior surface is shaped to contact spinous processes of the vertebral members.

4. The implant of claim 1, wherein the body includes a first height at the first stiffness level and a second shorter height at the second stiffness level.

5. The implant of claim 4, wherein the relief cut height decreases when the stiffness changes from the first stiffness level to the second stiffness level.

6. The implant of claim 1, further comprising a second relief cut contained within the body and extending from the exterior surface inward into the interior region, and a second resorbable material positioned within the second relief cut.

7. The implant of claim 6, wherein the second resorbable material is different than the resorbable material.

8. The implant of claim 1, further comprising an opening that extends through the interior region of the body from an inferior side to a superior side for bone growth between the first and second vertebral members.

9. The implant of claim 1, wherein the stiffness of the implant gradually decreases after the implant is initially inserted within the patient until the resorbable material is absorbed within the patient.

10. The implant of claim 1, wherein the relief cut extends between inferior and superior sides of the implant.

11. The implant of claim 1, further comprising a second non-resorbable material positioned within the relief cut with the resorbable material.

12. An implant for spacing apart vertebral members of a patient, the implant comprising:
a body with an exterior surface and an interior region;
a relief cut with inferior and superior sides that extend from the exterior surface inward into the interior region, the relief cut including a height defined between the inferior and superior sides; and
a resorbable material positioned within the relief cut;
the implant including a first overall stiffness based on a combination of body stiffness and material stiffness when initially inserted between the vertebral members, and a second overall stiffness based on the body stiffness a period of time after initial insertion with the second overall stiffness being less than the first overall stiffness.

13. The implant of claim 12, wherein the second overall stiffness is based solely on the body stiffness as the resorbable material is completely absorbed at the period of time after initial insertion.

14. The implant of claim 12, wherein a majority of the overall stiffness is caused by the material stiffness.

15. The implant of claim 12, wherein the implant includes a third overall stiffness after initial insertion and before the period of time, the third overall stiffness being less than the first overall stiffness and greater than the second overall stiffness.

16. The implant of claim 15, further including a second non-resorbable material positioned within the relief cut, the second overall stiffness based solely on the body stiffness and the second material stiffness.

17. The implant of claim 12, further comprising a second relief cut within the body containing a second resorbable material, the second resorbable material including a different absorption rate from the resorbable material.

18. An implant for spacing apart vertebral members comprising:
a body including an exterior surface and an interior region;
first and second relief cuts each contained within the body and including inferior and superior sides and extending from the exterior surface inward into the interior region; and
a first resorbable material positioned within the first relief cut, and a second resorbable material positioned within the second relief cut;
the implant gradually changing from a first stiffness when initially inserted between the vertebral members when the first resorbable material is within the first relief cut and the second resorbable material is within the second relief cut, to a second lesser stiffness after the first and second resorbable materials are absorbed.

19. The implant of claim 18, wherein the first and second resorbable materials include different absorption rates.

20. The implant of claim 18, wherein the first and second resorbable materials are completely absorbed with the body being solely responsible for the second stiffness.

21. The implant of claim 18, wherein the first and second relief cuts are positioned within the interior region of the body in an overlapping arrangement.

22. The implant of claim 18, wherein the first and second relief cuts extend into the interior region of the body from substantially opposing directions.

23. The implant of claim 18, wherein the first relief cut includes a different shape than the second relief cut.

24. The implant of claim 18, wherein the inferior and superior sides of the first and second relief cuts are substantially parallel.

25. An implant for spacing apart vertebral members comprising:
a body including an exterior surface and an interior region;
first and second relief cuts each contained within the body and including inferior and superior sides and extending from the exterior surface inward into the interior region; and
resorbable material positioned within the first and second relief cuts;
the body and the resorbable material acting in combination during an initial time period and comprising a first stiffness to space apart the vertebral members, and comprising a second stiffness after the initial time period to space the vertebral members.

26. The implant of claim 25, further including a second non-resorbable material positioned within at least one of the first and second relief cuts, the second material acting in combination with the body.

27. A method of spacing apart vertebral members, the method comprising:
positioning an implant comprising a body and a resorbable material between the vertebral members, the implant including an initial stiffness based on a combination of a body stiffness and a resorbable material stiffness;
positioning the implant for a resorbable material to become completely absorbed and the initial stiffness to decrease to a final stiffness based solely on the body stiffness.
